(19) United States
(12) Patent Application Publication
(10) Pub. No.: US 2012/0238857 A1
(43) Pub. Date: Sep. 20, 2012

(54) EXPANDABLE IMPLANTABLE PRESSURE SENSOR FOR INTRAOCULAR SURGERY

(75) Inventors: Vernon G. WONG, Menlo Park, CA (US); Douglas A. LEE, Menlo Park, CA (US)

(73) Assignee: ORTHOMEMS, Inc., Menlo Park, CA (US)

(21) Appl. No.: 13/232,300

(22) Filed: Sep. 14, 2011

Related U.S. Application Data
(60) Provisional application No. 61/383,624, filed on Sep. 16, 2010.

(51) Int. Cl. A61B 3/16

(52) U.S. Cl. 600/398

(57) ABSTRACT

Implantable pressure sensors provide for the direct measurement of IOP, for example following cataract surgery. The implantable sensors can couple to the capsule, for example separately from an IOL (intraocular lens), such that the sensor can be used with many commercially available IOLs. The surgeon can identify an IOL appropriate for the patient and place the implantable sensor when the patient has been identified as having glaucoma or at risk for glaucoma. The implantable sensor device can be implanted during IOL surgery.

### CROSS-REFERENCES TO RELATED APPLICATIONS

This application claims the benefit of provisional application No. 61/383,624 (Attorney Docket No. 41632-705.101), filed on Sep. 16, 2010, the full disclosure of which is incorporated herein by reference.

### BACKGROUND OF THE INVENTION

1. Field of the Invention

The present application relates generally to medical devices and methods, and more particularly to opthalmic implants capable of measuring pressure in the eye.

People like to see. The eye is a complex organ that allows a person to see his or her surroundings. The eye includes a cornea and crystalline lens that form an image on the retina of the eye. The retina of the eye senses the light image formed thereon and transmits neural signals via the optic nerve to the occipital cortex of the brain, such that the person can see and perceive his or her surroundings. Unfortunately, ocular diseases can compromise vision of the eye and may cause blindness in at least some instances.

Glaucoma is a major cause of blindness in the United States. In many instances, glaucoma related blindness can be prevented if caught and managed early. Glaucoma is usually associated with an increase in intraocular pressure (hereinafter “IOP”), that can result in damage to the retina of the eye. Because glaucoma is usually associated with an increase in IOP, periodic testing can be used to monitor glaucoma in order to prevent irreversible vision loss. For example, a person may undergo two to four exams per year in an ophthalmologist's office, although more examination may sometimes occur.

A significant clinical need exists to detect elevated IOP and/or fluctuations in IOP such that appropriate medical and surgical treatment can be delivered to control the patient's IOP and decrease vision loss. Unfortunately, at least some of the prior clinical techniques for measuring glaucoma may not detect elevated IOP, such that a patient can lose vision and may even become blind in at least some instances. For example, an ophthalmic exam may only measure IOP when the patient is in the eye clinic. In at least some instances, the patient may undergo an increase in IOP, for example a pressure spike, when the patient is away from the clinic. As such pressure spikes may not be detected, the patient may not receive treatment in time to mitigate vision loss. Further, at least some patients may not be able to visit the eye clinic on a strict regular basis, for example elderly patients and children, such that an increase in IOP may not be detected in a timely manner so as to prevent vision loss in at least some instances. Also, in at least some instances a patient may simply forget to take his or her medicine, such that the patient fails to follow the prescribed treatment.

Although measurements with an external IOP sensor can be helpful, these devices that measure pressure of the eye with an external sensor are somewhat indirect and can be inaccurate in at least some instances, such that the measured IOP may differ from the actual pressure inside the eye. In at least some instances, clinically available IOP sensors determine the IOP based on the externally measured pressure. For example, the IOP sensor can measure pressure of the eye on the external surface of the cornea, for example with applanation or indentation of the cornea. The externally sensed pressure of the eye can be used to determine the IOP of the eye based on assumptions about the anatomy and characteristics of the patient's eye. Such assumptions can lead to errors in the indirectly measured IOP when the anatomy of the patient deviates from the assumed normal anatomy and characteristics in at least some instances. For example, external IOP measurements can be affected by scleral rigidity influenced by topical anti-glaucoma drug therapy so as to induce errors in the externally measured IOP in at least some instances. As a result, in at least some instances a patient may not receive appropriate treatment.

Many patients who receive intraocular lenses (hereinafter “IOLs”) can be at risk for glaucoma, for example patients who undergo cataract surgery. Recent clinical studies indicate that the coincidence of glaucoma and cataract can be from about 10 to 20% of patients receiving IOLs in at least some patient populations. However, at least some of the prior IOP sensors may not be well suited for combination with IOLs and can be more invasive than would be ideal. Also, although IOLs having pressure sensors have been proposed, the fitting of IOLs to the eye can be complex and such lenses may not be well suited for use with at least some patients in at least some instances. Although prior IOLs can provide successful results, in at least some instances the implanted IOL may provide a less than ideal refractive result. For example, the centration of an IOL within the eye can be less than ideal in at least some instances. Placing a prior IOL having a pressure sensor can limit the available IOL choices such that the patient visual outcome may be less than ideal in at least some instances. Also, at least some of the proposed prior IOP sensors may not fit at least some eyes as well as would be ideal in at least some instances.

It would be helpful to provide improved methods and apparatus that overcome at least some of the above shortcomings, for example by providing implantable IOP devices capable of at least daily direct measurement of IOPs by providing implantable IOP devices that are less invasive than prior devices and, by providing implantable IOP devices that are compatible with many or all IOLs, such that the improved device can be implanted in patients with glaucoma and cataracts. Ideally, such methods and apparatus can be implanted in the eye quickly and easily in an outpatient environment, and such that many patients can receive the benefit of direct monitoring of IOP.

2. Description of the Background Art

The disclosure of U.S. Pat. Nos. 5,005,577 and 6,796,942 are relevant to the present invention. WO 2011/035228 and WO 2011/035262 are commonly owned and have common inventorship with the present application. The disclosures of these two commonly owned applications are incorporated herein by reference.

### BRIEF SUMMARY OF THE INVENTION

Embodiments of the present invention provide improved methods and apparatus for the direct measurement of IOP such as following cataract surgery. Implantable IOP sensor devices can be placed in the eye with or without an IOL but are particularly well suited for implantation together with an IOL. The implantable sensor devices of the present invention comprise an expandable coil structure which can couple to the capsule or other tissue structure separately from the IOL such that the sensor can be used with many commercially available IOLs, allowing the surgeon to implant an IOL appropriate for vision correction of the patient and to implant the implantable sensor only if the patient has glaucoma or is at risk for glaucoma. The coil structure expands radially when placed in the eye so as to urge radially outward against a tissue structure of the eye and fix the location of the implantable device in the eye and optionally provide support to the tissue structure such as with radially tensioning (eg to circumferentially reinforce a damaged capsular bag), and in many embodiments so as to center the expandable loop antenna about the optical axis of the eye. The area within the radially expandable coil will also provide a location for optional implantation of the IOL, where the implantable sensor may be implanted before or after implantation of the IOL. By implanting the implantable sensor devices of the present invention during IOL surgery, the invasiveness of the procedure is minimized, allowing many patients to receive benefit from direct measurement of IOP together with vision correction with an IOL that is best suited to the patient.

In many embodiments, the expandable coil structure of the expandable implantable sensor device urges radially outward so as to engage the tissue structure of the eye when implanted, such that expandable implantable sensor device can accommodate many eye sizes. The expandable coil structure preferably comprises a radially expandable loop antenna capable of expanding radially to an outer dimension of at least about 13 mm across, often at least about 15 mm across, typically being in the range from 8 mm to 15 mm, such that the radially expandable loop antenna of the expandable coil structure urges radially outwardly against the capsular bag or other tissue structure of the eye when implanted. The expandable coil structure comprises the radially expandable loop antenna or other structure that expands radially outwardly to fit the eye and a pressure sensor, such as a toroidal or other coil connected in series to a pressure sensor to determine the IOP of the eye based on the resonant frequency of the pressure sensor coupled to the radially expandable loop antenna connected and the coil (a variety of other pressure sensors would also be suitable). The loop antenna of the expandable coil structure can expand radially outward so as to tension a tissue structure of the eye such as the lens capsule (capsular bag), and so as to hold the expandable implantable sensor device in place when the expandable loop antenna urges radially outward and engages the tissue structure. The expandable loop antenna of the expandable coil structure can also center the implantable sensor device about the optical axis of the eye, and can also help to restore and center tissue structures of the eye, such as the lens capsule, when neighboring tissue is at least partially damaged during surgery, for example when zonules of the lens capsule are damaged during surgery. Such implantable structure will be suitable for permanent implantation but will also be removable where long term pressure monitoring is necessary.

In a first aspect, embodiments provide methods of treating an eye having a tissue structure where the interior of an eye, usually of a lens capsule, is accessed. A lens (IOL) is inserted into the interior of the eye. An implantable (IOP) sensor device is also inserted into the interior eye, either before, after, or simultaneously with insertion of the lens. The implantable sensor device has a pressure sensor and an electrically conductive coil structure electrically and mechanically coupled to the pressure sensor, wherein the electrically conductive coil structure physically engages (couples to) the tissue structure of the eye to hold the implantable sensor within the eye. The IOL can be placed in the anterior chamber of the eye, the posterior chamber of the eye, or the lens capsule of the eye. The sensor device can be placed in the anterior chamber of the eye, the posterior chamber of the eye, or the lens capsule of the eye, for example adjacent to the IOL, preferably where the loop antenna circumscribes the lens.

In another aspect, embodiments of the present invention provide methods of treating an eye having lens, in which the lens has a capsule. An IOL is inserted within the capsule, and the IOL comprises a haptic to expand against the capsule. An implantable sensor device is implanted within the capsule, in which the implantable sensor device has a pressure sensor and an electrically conductive coil structure coupled to the pressure sensor. The electrically conductive coil structure physically engages (couples to) the capsule so as to hold the implantable sensor within the capsule.

In another aspect, embodiments provide implantable devices for measuring an intraocular pressure of an eye having a lens, in which the lens has a capsule. The device comprises a pressure sensor and an expandable electrically conductive coil structure coupled to the pressure sensor. The expandable coil structure comprises a resilient material to urge the coil structure against the capsule to hold the coil structure and the pressure sensor within the capsule. The expandable coil may be self-expanding, i.e. formed from a material with sufficient elasticity to allow the fully expanded coil to be constrained to a low profile to facilitate introduction into the capsular bag (after suitable opening and preparation) or other structure of the eye. Suitable materials include using stainless steels and shape memory alloys. The expandable coil will also usually be adapted to act as receivers/transmitters for externally communicating with the pressure sensor, for example to allow frequent or real time monitoring of the intraocular pressure. Alternatively, the coils may be sufficiently malleable and/or have joints, serpentine or zig-zag sections, or otherwise be adapted to permit expansion by a balloon or other expandable delivery tool.

### BRIEF DESCRIPTION OF THE DRAWINGS

FIG. 1 shows an eye suitable for incorporation with an IOL and an implantable sensor, in accordance with embodiments of the present invention;

FIG. 2A shows an eye having an implantable pressure sensor placed in the capsular bag formed during cataract surgery, in accordance with embodiments;

FIG. 2B shows the support, coil structure and loop trace of the implantable apparatus of FIG. 2A, in accordance with embodiments;

FIGS. 2C1 and 2C2 show side and end views, respectively, of the apparatus as in FIGS. 2A and 2B having an elongate narrow profile incision for insertion through an incision in the cornea of the eye in which the apparatus has been folded to the elongate narrow profile configuration for insertion;

FIG. 2C3 shows side view of the apparatus as in FIGS. 2A and 2B having an elongate narrow profile configuration for insertion through an incision in the cornea of the eye in which the apparatus has been rolled to the elongate narrow profile configuration for insertion;

FIG. 2C4 shows an inserter having a lumen sized to receive the implantable sensor device having the elongate narrow profile configuration for insertion through the incision, in accordance with embodiments;

FIG. 2D shows the eye having an implantable pressure sensor comprising a C-shaped coil structure placed in the capsular bag formed during cataract surgery so as to expand radially from the optical axis so as to fit the capsular bag, according to embodiments of the present invention;

FIG. 2E shows the support, coil structure and loop trace of the implantable device of FIG. 2D in accordance with embodiments;

FIG. 2F shows a side view of the apparatus as in FIGS. 2D and 2E having an elongate narrow profile configuration for insertion through an incision in the cornea of the eye in which the apparatus has been unfolded to the elongate narrow profile configuration for insertion;

FIG. 2G shows the eye having an implantable pressure sensor device comprising a radially expandable coil structure for placement in the capsular bag formed during cataract surgery so as to expand radially from the optical axis so as to fit the capsular bag, in accordance with embodiments of the present invention;

FIG. 2H shows an inserter apparatus comprising a cartridge to insert the implantable sensor through the incision in the cornea, in accordance with embodiments;

FIG. 2I shows an inserter tool for use with an inserter cartridge as in FIG. H, in accordance with embodiments;

FIG. 2J shows an IOL for use with the implantable sensor apparatus and the inserter, in accordance with embodiments;

FIG. 3 shows components of a telemetry system comprising the implantable sensor, in accordance with embodiments;

FIG. 3A shows components of an implantable MEMS pressure sensor for use with the implantable apparatus, in accordance with embodiments of the present invention;

FIG. 4A shows components of an antenna reader, in accordance with embodiments of the present invention;

FIG. 4B shows a hand held antenna reader with components similar to the antenna reader as in 4A; and

FIG. 5 shows a method of treating a patient with an implantable pressure sensor apparatus implanted with an IOL during surgery, in accordance with embodiments.

FIG. 6 illustrates an additional exemplary implantable pressure sensor device constructed in accordance with the principles of the present invention.

### DETAILED DESCRIPTION OF THE INVENTION

Embodiments of the present invention described herein can addresses a significant clinical need for patients having glaucoma, and the expandable implantable sensor device as described herein is well suited for combination with intraocular lenses such as IOLs implanted with cataract surgery. The construction and shape of the device allows the device to be easily implanted through an incision to implant an IOL during cataract surgery such that a person receiving an IOL can also receive direct measurement of IOP, for example a patient who has glaucoma or may be at risk for glaucoma.

The measurements may be intended to monitor pressure within predefined ranges and/or may be intended to monitor fluctuations over time. Many embodiments described herein provide a direct measurement of intraocular pressure. The intraocular pressure can be measured as often as practical, for example with a hand held reader coupled to the implanted device. The measurements can be made with sufficient frequency so as to determine the presence of diurnal IOP curves and so as to detect IOP peaks and pressure spikes. For example, the measurements can be generated hourly for the first few days following surgery, and then with decreasing frequency as the patient's pressure stabilizes. The direct IOP measurements can be made at many locations including at home or a doctor's office. The hand held reader may automatically forward the patient information to the treating physician, such that the physician can monitor the patient remotely. The readings can be continued indefinitely, or the device may be removed from the eye and the readings stopped after sufficient pressure data have been collected.

As used herein, like numerals refer to like elements.

As used herein, the anterior segment of the eye encompasses the anterior chamber of the eye and the posterior chamber of the eye.

The implantable device component can be interrogated with an antenna and reader circuitry configured to determine IOP of the eye in response to the signal transmitted from the implanted device. The reader circuitry is coupled to a computer processor configured to a computer processor to store and transmit data.

In many embodiments, the implantable device comprises a MEMS based pressure sensor for use with the treatment of glaucoma that facilitates accurate measurement/monitoring of patient IOP of the anterior chamber. Many embodiments utilize MEMS and wireless technology that can provide direct, continuous and real-time data on IOP. The implant may comprise a coil structure joined to a pressure sensor encapsulated in a medical-grade biocompatible material. The coil structure can be configured with an elongate narrow profile configuration and inserted through an IOL incision by a surgeon, such that the tip of the device with the attached sensor comprising the pressure responsive transducer sits inside the anterior chamber of the eye. In some cases, commercially available pressure sensors, such as those available from VTI Technologies Oy (Finland), such as the Capacitive Absolute Pressure Sensors series, may be suitable.

The implant can be removed, for example in case of adverse events or when pressure monitoring is no longer desired.

Following implantation, direct IOP measurements can be obtained real-time and continuously with a data acquisition unit that wirelessly interrogates the implanted sensor, and includes hardware/software to control an external antenna and monitor pressure fluctuation patterns for normal/pathological conditions. The IOP measurement comprises a direct measurement as the transducer of the pressure sensor is implanted in the target tissue of interest, for example the anterior segment.

The direct IOP measurement data can be used in many beneficial ways. For example, the direct IOP measurement data can be used to trigger an alarm for the patient with the hand held reader, and the data can be transmitted to a remote server and to the office of the treating physician. The data at the remote server can be analyzed, for example mined, to determine statistical trends and analysis and algorithm development. The algorithm can be embodied in instructions of a computer program of the server. The data at the physician's office can be used by the physician to monitor the patient.

The implantable MEMS pressure sensor device and external telemetry may comprise components as described in U.S. Pat. No. 6,682,490, the full disclosure of which is incorporated by reference and suitable for combination in accordance with some embodiments of the present invention described herein.

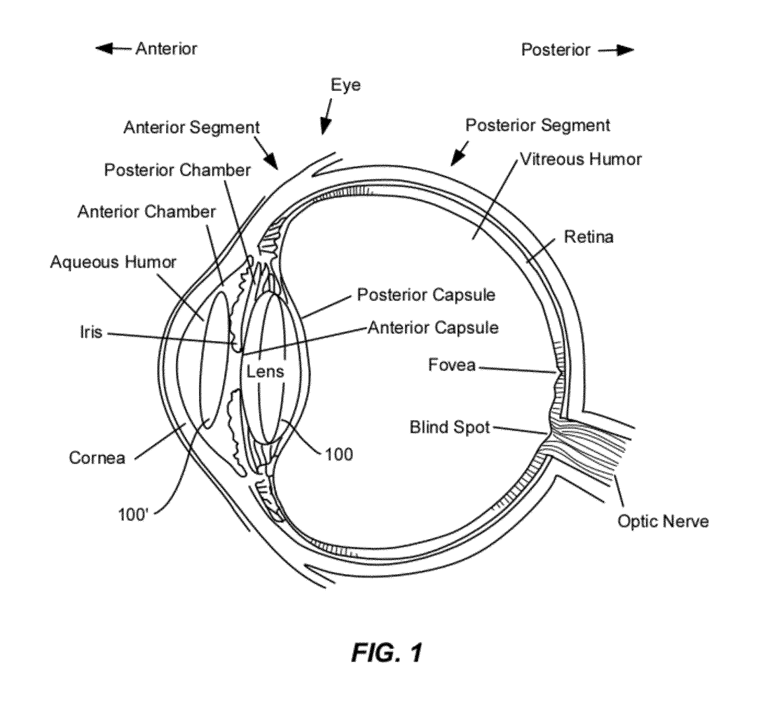

FIG. 1 shows an eye suitable for surgical implantation of the implantable sensor and IOL, as described herein. The eye generally comprises an anterior orientation toward the front of the eye and a posterior orientation toward the back of the eye. The eye comprises a cornea and lens that refract light so as to form an image on the retina of the eye. The retina comprises a fovea comprising light sensitive cones to detect light color sensitivity and high visual acuity. The retina also comprises a blind spot where the optic nerve couples to the retina. An iris is disposed over the lens and responds to light so as to dilate in darkness and constrict in bright light, such that the intensity of light striking the retina can be increased and decreased, respectively. The eye comprises an anterior segment and a posterior segment, with the lens disposed there between. The anterior segment comprises an aqueous humor and the posterior segment comprises a vitreous humor. The posterior chamber of the eye extends between the iris and the anterior capsule of the lens and comprises the aqueous humor. The anterior segment comprises the posterior chamber. The liquid of the eye generally drains from the posterior segment to the anterior segment and out Schlemm's canal so as to maintain intraocular pressure.

The lens of the eye comprise a lens capsule, a cortex and a capsular bag located around the cortex and nucleus. A cataract may form in the nucleus or the cortex so as to scatter light that can degrade vision. With cataract surgery, the capsular bag can be cut with a capsulorhexis incision and the cortex and nucleus removed from the capsular bag. The IOL can be inserted within the capsular bag. Alternatively, the IOL may comprise a phakic IOL used with the natural lens of the eye, and the phakic IOL can be configured for placement in the anterior chamber or the posterior chamber of the eye.

Schlemm's canal, also known as canal of Schlemm or the scleral venous sinus, comprises a circular channel in the eye that collects aqueous humor from the anterior segment and delivers the liquid of the aqueous humor into the bloodstream. The canal comprises an endothelium-lined tube. On the inside of the canal, nearest to the aqueous humor, the canal is covered by the trabecular meshwork, and this region contributes to outflow resistance of the aqueous humor.

With glaucoma, the drainage of aqueous liquid from the anterior chamber is less than ideal such that IOP can increase in the anterior chamber.

The expandable implantable pressure sensor 100 as described herein can be placed at one or more of many locations of the eye such as within the anterior chamber, the posterior chamber, or the capsular bag, for example. As shown in FIG. 1, the implantable sensor can be implanted at the ocular location with a lens such as one or more of an IOL place in the capsular bag, an IOL placed in the posterior chamber, or an IOL placed in the anterior chamber. For example, expandable implantable sensor 100 can be placed in the posterior chamber with an IOL placed in the posterior chamber. Alternatively, expandable implantable sensor 100 can be placed in the anterior chamber with an IOL placed in the anterior chamber. When a patient undergoes cataract surgery to remove the lens of the eye, an expandable implantable sensor 100 can be placed in the capsular bag with an IOL placed in the capsular bag during cataract surgery. The expandable pressure sensor 100 could alternatively be placed within a separate tissue structure of the eye from the lens, such as in the posterior chamber when the lens is implanted in the cornea. The expandable implantable pressure sensor 100 can be sized to couple to the eye at the placement location as described herein.

The expandable implantable pressure sensor 100 can be used with many commercially available implantable lenses such as intra-corneal lenses implanted in the cornea of the eye, implantable collamer lenses. Examples of commercially available lenses suitable for combination with expandable implantable sensor 100 include phakic IOLs placed in the posterior chamber such as the Visian ICL™ commercially available from STAAR® Surgical Company, intraocular lenses placed in the lens capsule such as the Tecnis™ IOL commercially available from Abbott Medical Optics. The expandable implantable sensor 100 can be combined with lenses composed of one or more of many materials such as silicon, plastic, acrylate, soft acrylate, collagen, or polymer comprising collagen such as collmer, for example.

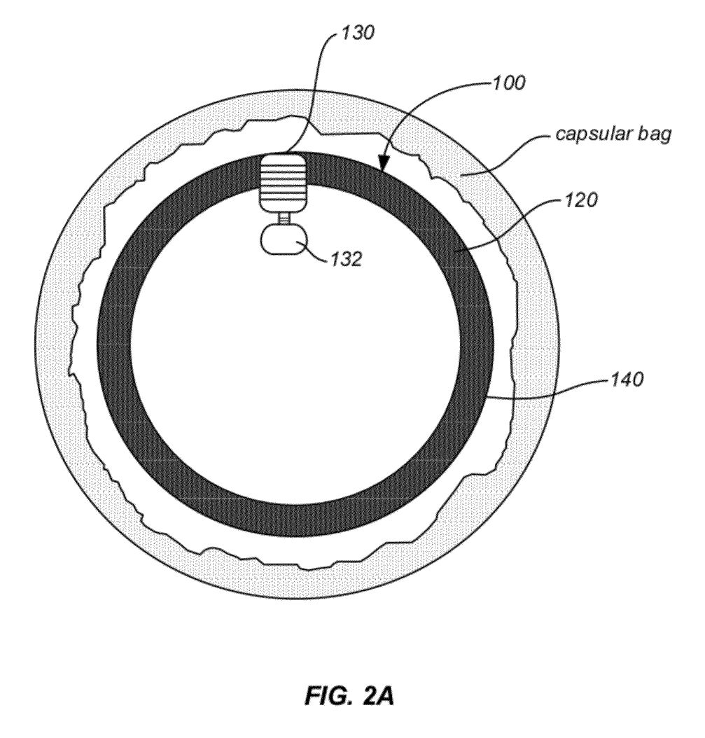

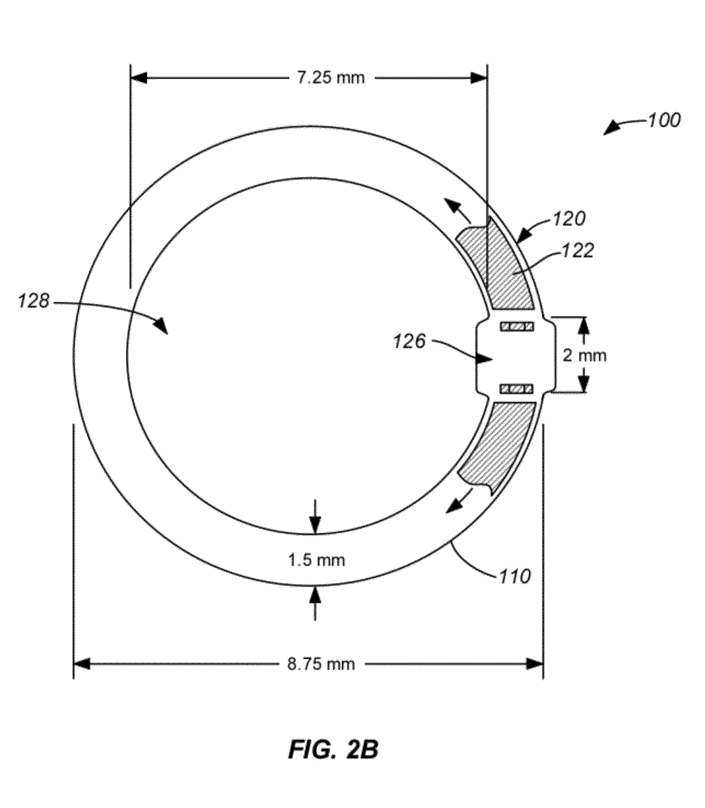

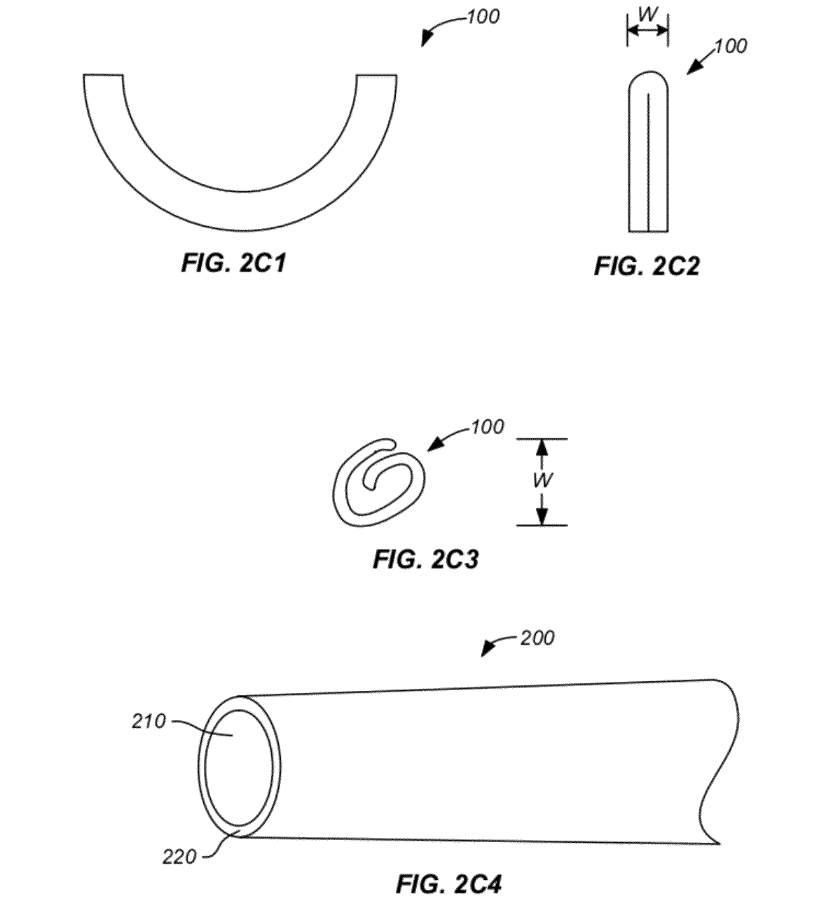

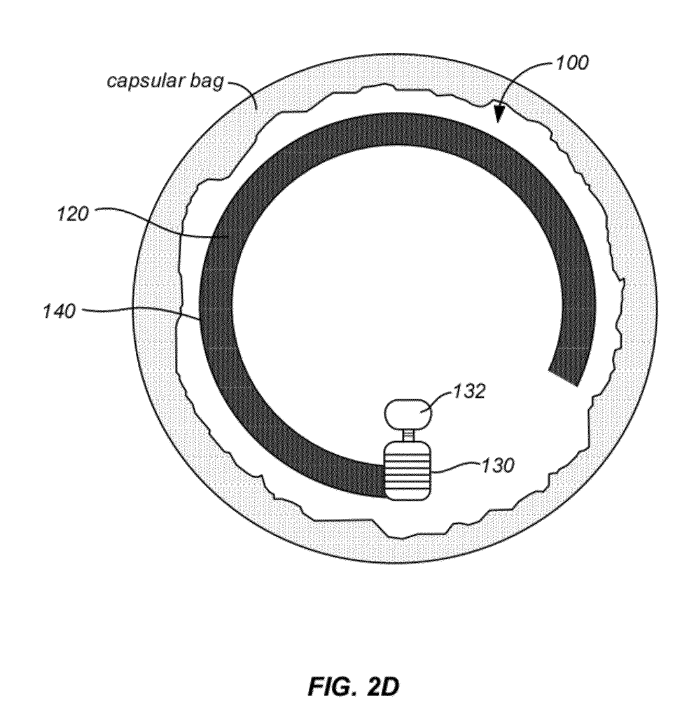

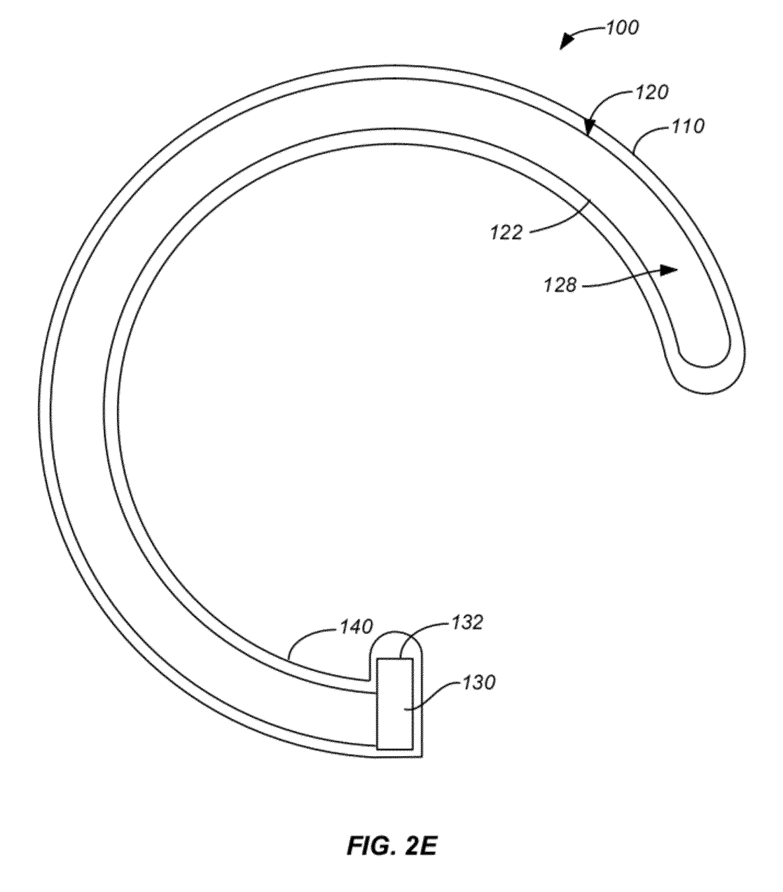

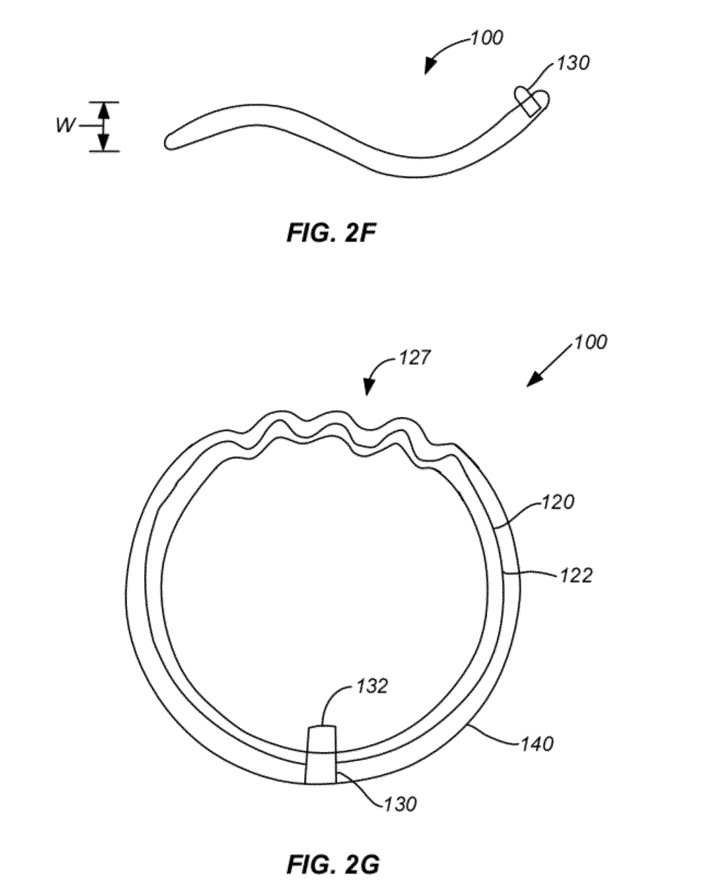

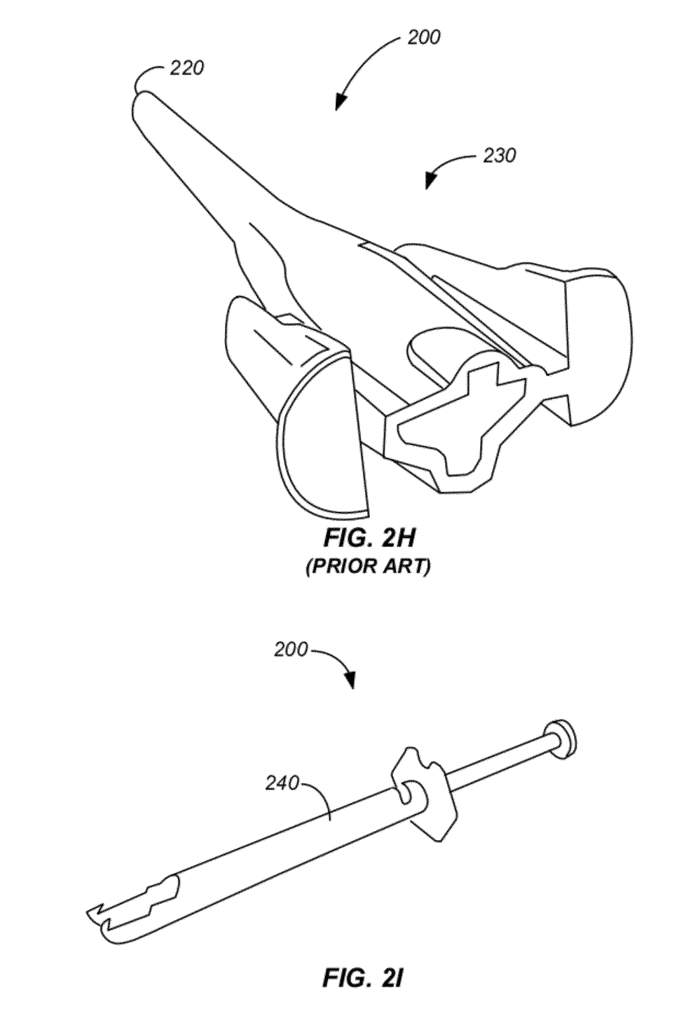

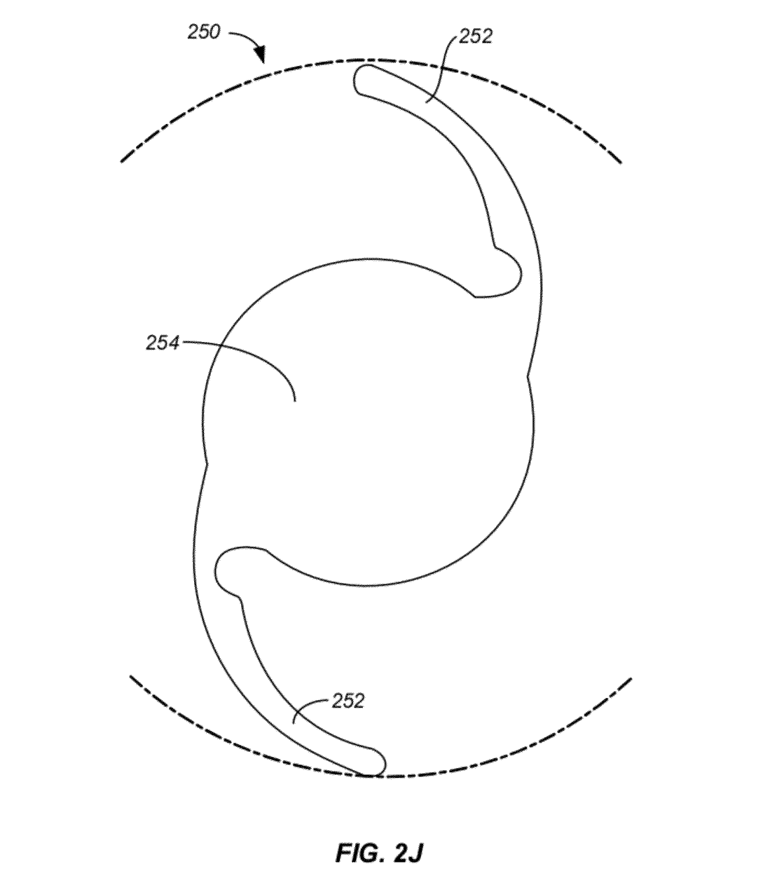

FIG. 2A shows an implantable pressure sensor device 100 placed in the capsular bag formed during cataract surgery. The implantable pressure sensor device 100 has an expanded wide profile configuration 104 when placed in the capsular bag. Device 100 comprises a coil structure 120 coupled to an implantable MEMS pressure sensor 130. The coil structure 120 may comprise an inductive antennae loop 122 (FIG. 2B) for example a single loop antenna extending substantially around the pupil of the eye. The coil structure 120 may comprise an additional coil such as a toroidal coil having a ferrite core coupled to the MEMS pressure sensor. The implantable MEMS pressure sensor 130 may comprise a pressure sensitive capacitor 132 that may be oriented inwardly toward an optical axis of the eye corresponding to a center of the pupil. The IOP of the eye may be determined based on the resonant frequency of the coil structure coupled to the MEMS pressure sensor. A soft coating material such 140 can cover the MEMS pressure sensor 130 and coil structure 120. Although the long axis of the pressure sensor device 100 is oriented radially, the device could be turned 90° and/or inclined relative to the plane of the coil to limit intrusion and/or improve exposure to the aqueous pressure.

FIG. 2B shows the support, coil structure and loop trace 126 of the implantable pressure sensor of FIG. 2A. An antenna loop 122 of the coil structure 120 can extend annularly within an annular support 110 with trace deposited on the support. Annular support 110 may comprise a flex PCB material such as polyimide or other flexible material, and antenna loop 122 can extend substantially around the annular support 110. The annular antenna loop 122 can define an inductive area 128 for signal transmission and may comprise an inner dimension across. The inner dimension across the antenna loop can be at least about 6 mm across, for example so as to define an area of about 30 mm2, and so as to allow a clear aperture of at least about 6 mm for vision through the IOL. The loop may have a larger distance across, for example at least about 7 mm so as to define an area of at least about 40 mm2, for example. The outer dimension of the annular loop structure can be about 9 mm across so as to fit within the capsular bag. Exemplary dimensions are provided on FIG. 2B.

As the size of the capsular bag can very among individuals, the physician can be provided with a plurality of implant devices where each device has a different outer dimension to fit the capsular bag of the individual patient. For example, the physician can be provided with a plurality of sensor devices having at least three sizes or more, e.g. 8 mm, 9 mm, 10 mm, 11 mm, and 12 mm, for example. The physician can determine the size of the tissue structure such as the capsular bag and identify the size of sensor device to implant in the tissue structure such as the capsular bag.

The coil structure 120 may comprise a coil such as a toroidal coil 126 coupled to the single loop antenna 122, for example connected in series with the single loop antenna to the pressure sensor, such that the combined inductance is equal to the inductance of the loop antenna 122 and toroidal coil 126 so as to lower the resonant frequency. The toroidal coil 126 may have an inductance greater than the inductance of the single loop coil so as to maintain the resonant frequency of the circuit when the area of the single loop coil changes, for example with embodiments having an expandable coil structure as described herein. The support structure 120 may comprise conductive pads coupled to the traces of the loop antennae 122 that define coil and space to receive the MEMS pressure sensor 130 and toroidal coil 126.

FIGS. 2C1 and 2C2 show side and end views, respectively, of the apparatus as in FIGS. 2A and 2B having an elongate narrow profile configuration for insertion through an incision in the cornea of the eye in which the apparatus has been folded to the elongate narrow profile configuration for insertion. The width W of the narrow profile configuration may correspond to the size of the incision in the cornea for IOL surgery, for example the incision can be about 3 mm or less and the narrow profile configuration sized to fit through the incision of 3 mm or less.

FIG. 2C3 shows side view of the sensor device 100 as in FIGS. 2A and 2B having spinal configuration with a reduced width for insertion through an incision in the cornea of the eye in which the apparatus.

FIG. 2C4 shows an exemplary inserter 200 having a distal tip 220 sized for insertion through an incision in the eye and comprising a lumen 210 sized to receive the sensor device in a reduced width configuration and having an elongate narrow profile configuration, usually tapered, for insertion through the incision. The inserter may comprise components of a commercially available IOL inserter. The dimension across the lumen may comprise a diameter of no more than about 3 mm across, for example.

FIG. 2D shows an implantable pressure sensor device 100 comprising a C-shaped coil structure 120 placed in the capsular bag formed during cataract surgery so as to expand radially from the optical axis so as to fit the capsular bag. The C-shaped coil structure 120 can expand radially away from the optical axis of the eye so as to fit the capsular bag such that C-shaped coil structure 120 can fit at least about 90% of patients, for example at least about 95% of patients. The radially expandable coil structure 120 can tension the capsular bag annularly, for example when at least a portion of the zonules have been destroyed such that C-shaped coil structure 120 can be placed prior to the IOL in at least some instances. The C-shaped coil structure comprises a first end and a second end, and the MEMS pressure sensor 130 can be located on the first end or the second end (as illustrated), or for example equidistant between the first end and the second end (not illustrated).

FIG. 2E shows the support, coil structure and loop trace of the implantable apparatus of FIG. 2D. The antenna loop 122 of the coil structure 120 can extend along an outer portion of annular support 110 with trace deposited on the support and extend along an inner portion of support 110 so as to define area 128 with a radial separation distance between the inner annular portion and the outer annular portion. The radial separation distance should be at least about 1 mm, for example at least about 2 mm, such that inductive area 128 is sufficient for signal transmission. The annular support 110 may comprise a flex PCB material such as polyimide or other flexible material, and antenna loop 122 can extend substantially around an inner boundary of the annular support 110 and an outer boundary of the annular support 110 such that the inductive area 128 defined with the loop remains substantially constant when the C-shaped coil structure expands to fit the capsule and such that the resonant frequency is substantially maintained. The toroidal coil in pressure sensor 130 can be coupled to the inductive antenna loop 122 so as to maintain the resonant frequency as described above. The soft coating 140 can cover the support 110, coil structure 120 and sensor 130 as described above.

FIG. 2F shows a side view of the apparatus as in FIGS. 2D and 2E having an elongate narrow profile configuration for insertion through the incision in the cornea of the eye in which the apparatus has been unfolded to the elongate narrow profile (width W) configuration for insertion, for example through the inserter as described herein.

FIG. 2G shows the implantable pressure sensor device 100 comprising a radially expandable coil structure for placement in the capsular bag formed during cataract surgery so as to expand radially from the optical axis so as to fit the capsular bag. The expandable loop antenna of the expandable coil structure may comprise an expandable segment 127 such as undulations, coils, or slack, so as to allow the loop to expand and fit the eye.

FIG. 2H shows an inserter apparatus 200 comprising a cartridge 230 to insert the implantable sensor through the incision in the cornea. The inserter apparatus 200 may comprise a commercially available IOL inserter such as Monarch™ II IOL Delivery System commercially available from Alcon Laboratories, the insertion system for use with the Tecnis™ 1 Piece IOL commercially available from Abbot Medical Optics, or the SofPort® Advanced Optics Aspheric Lens System commercially available from Bausch and Lomb, although many commercially available IOL inserters can be used.

FIG. 21 shows an inserter tool 240 for use with an inserter cartridge 230 as in FIG. 2H. The implantable sensor device 100 can be placed in the loading zone of the cartridge 230 and coupled to the inserter tool 240. The plunger rod can be advanced to insert the sensor device 100 in the capsule.

FIG. 2J shows an IOL 250 having haptics 252 for use with the implantable sensor apparatus and the inserter. The IOL 250 may comprise SA60AT Acrysof™ commercially available from Alcon, the Tecnis™ 1 Piece IOL, or the Akreos AO Aspheric IOL commercially available from Bausch and Lomb, although many commercially available IOLs can be used.

System Components and Function

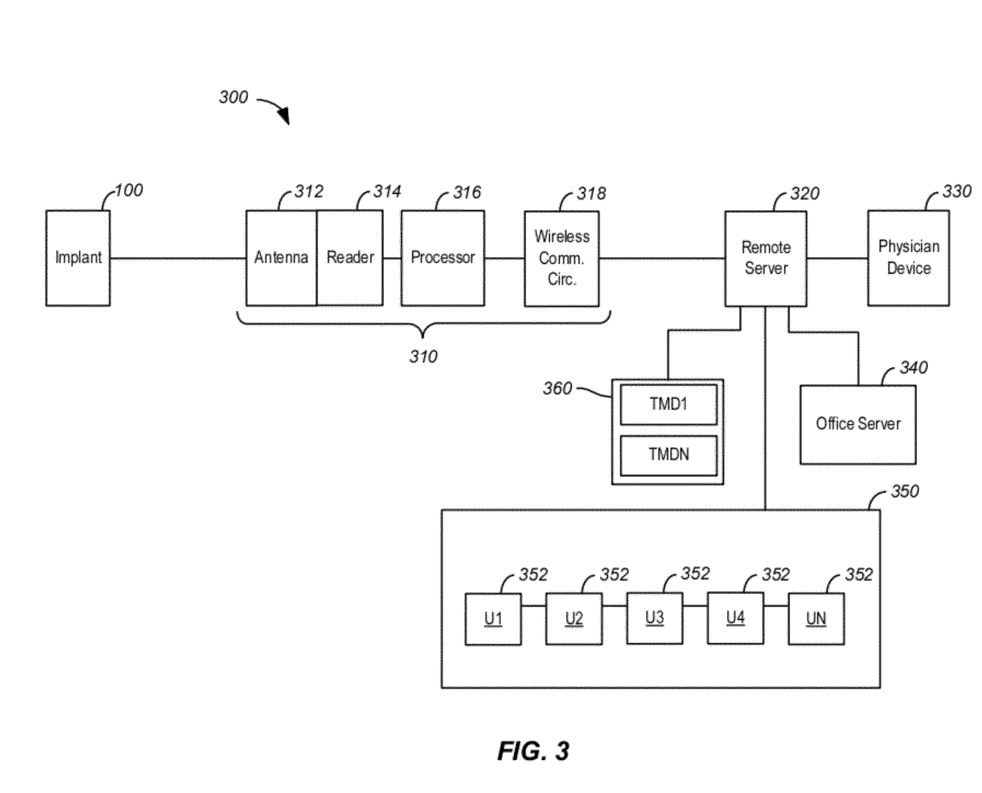

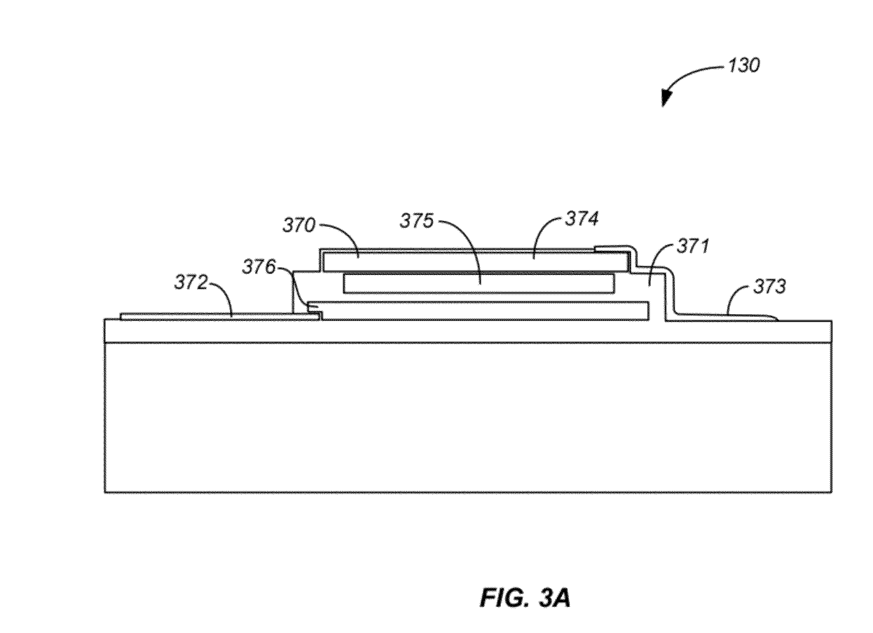

FIG. 3 shows components of a telemetry system 300 comprising the implantable sensor. The wireless communication based pressure sensing system may comprise several components. The implantable sensor 10 is configured to couple to an external reader 310, for example an antenna/reader, to determine the resonant frequency of the pressure sensitive capacitor and inductor circuit. The antenna/reader comprises an antenna 312 and reader circuitry 314 to determine the resonant frequency of the implanted sensor. The external reader 310 is configured to determine the patient IOP based on the directly measured pressure within the eye and the external atmospheric pressure. As atmospheric pressure can fluctuate approximately +/−10 mm of Hg and may also change with the elevation of the patient, the accuracy of the patient IOP reported to the physician and patient can be improved substantially by determining the reported IOP based on the IOP measured directly with the implanted pressure sensor and the atmospheric pressure external to the eye.

The external reader 310 can be configured in many ways to determine the IOP of the patient based on the directly measured IOP and the atmospheric pressure. For example, the external reader 310 may comprise an atmospheric pressure sensor to determine the IOP reported to the physician and the patient based on the IOP measured directly with implanted sensor and the local atmospheric pressure. Alternatively or in combination, the external reader 310 may have two way communication with an external weather site to determine the atmospheric pressure from the external site. For example, the external site may comprise a local weather station or web site having a corresponding internet address, and the atmospheric pressure where the patient is located can be determined based on one more of postal zip code, latitude and longitude, or global positioning system coordinates. The external reader 310 may comprise circuitry to determine the location of the patient and use the patient position in formation to determine the pressure where the patient is located based on meteorological weather information. The global positioning coordinates of the patient can be determined in many ways, for example with location based on a cellular phone connection of the external reader 310 or based on GPS circuitry of the reader 310.

Atmospheric pressure associated with weather can fluctuate slowly and on the order of +/−about 10 mm of Hg, such that correction of measured patient IOP based on commercially available meteorological information can be sufficient to provide accurate determination of the patient IOP when combined with the directly measured IOP. Also, by determining the location of the patient, fluctuations in atmospheric pressure associated with the elevation where the patient is located can determined and used to determine the patient IOP. For example, the IOP reported to the physician and patient can be determined by subtracting the barometric pressure at the location and elevation of the patient from the directly measured IOP to determine the corrected IOP reported to the physician and patient. The elevation of the patient can be determined based on the location of the patient, for example when the patient is located at a city near sea level or a city in the mountains. The rate of change in patient location can also be used, for example when the patient flies and location changes quickly.

The adjusted IOP (AIOP) for patient reporting and can be determined in many ways based on the directly measured internal IOP and externally measured atmospheric pressure. For example, the adjusted IOP (ΔIOP) may comprise a differential IOP determined by subtracting the external atmospheric pressure (ATP) from the internally measured IOP (IMIOP) with the equation (AIOP)=(ΔIOP)=(IMIOP)−(ATP).

Although a calculation is shown, the adjusted IOP can be determined in many ways, for example with a look up table stored in a processor.

The antenna/reader is coupled to a processor 316 comprising a computer readable medium having instructions of a computer program embodied to determine the intraocular pressure, for example with a look up table, in response to the resonant frequency and the local atmospheric pressure. The at least one processor can be coupled to the Internet with wired or with wireless communication circuitry and transmit the patient data to a server 320 located remote from the patient. Alternatively or in combination, the patient data can be transmitted to a treating physician for evaluation of the patient. For example, the data can be transmitted to a server located at the treating physicians office. The data can also be transmitted to the physician with wireless cellular communication, for example to a handheld physician communication device such as a pager, iPhone™, Blackberry™, such that the physician can evaluate the status of the patient and may adjust treatment of the patient accordingly.

The external antenna/reader 310 may comprise a hand-held ambulatory device comprising the atmospheric pressure sensor, the processor 316 and the wireless communication circuitry such that the patient can transmit measurement data with the wireless communication circuitry. For example, the wireless communication circuitry may comprise one or more of Wi-Fi circuitry or cellular circuitry, such that the patient user can measure and transmit data to the central server when the patient is mobile. The handheld ambulatory external reader 310 may comprise circuitry similar to hand held communication devices such as pagers and smart phones, for example the iPhone™ or the Blackberry™ smart phones. The handheld external reader 310 may comprise instructions of a computer readable program embodied on a tangible medium to determine the IOP reported to the physician based on the IOP measured directly with implanted sensor and the atmospheric pressure. For example, the atmospheric pressure can be determined based on the location and elevation of the patient and local barometric pressure, as described herein.

The remote server 320 may comprise data from many patients and comprise instructions of a computer program embodied on a programmable memory, such that the data from many patients can be combined and analyzed. For example, the server may comprise a data center where data are analyzed and physicians can share patient data. Alternatively or in combination, the patient data can be transmitted to a treating physician for evaluation of the patient. For example, the data can be transmitted to a server 340 located at the treating physicians office. The data can also be transmitted to the physician with wireless cellular communication, for example with to a handheld physician communication device 330 such as a pager, iPhone™ smart phone, or Blackberry™ smart phone, such that the physician can evaluate the status of the patient and may adjust treatment of the patient accordingly.

The system 300 may comprise a processor system, and the processor system may comprise two or more of the processor located with the patient, the remote server, the server located at the physician office, and the hand held physician communication device. The remote server comprises processor comprising a computer readable medium having instructions of a computer program embodied thereon so as to store patient data with a database.

The hand held communication device 330 can be configured such that the physician can transmit treatment instructions for patient treatment so as to close the loop of the treatment for the patient, for example with changes to medication or requesting a patient examination. The remote server comprises processor comprising a computer readable medium having instructions of a computer program embodied thereon so as to store patient data with a database. The remote server may also forward treatment instructions from the physician device 330 to the patient device 310.

The instructions from the handheld physician communication device allow the physician to direct patient treatment. For example, the physician can instruct the patient to come in for a visit, for example to assess the status of the patient need for additional surgical intervention. The physician may adjust the patient medication, for example increase the patient medication. The physician may set a target IOP for the patient based on the clinical assessment of the patient. Some patient who have lost vision can be more sensitive to IOP than those who have not, such that the physician may set the target IOP for a patient with vision loss lower than a patient who has not lost vision. For example, the physician can set the target IOP for a patient with vision loss at 12 mm Hg, and the target IOP for a patient with no vision loss at 21 mm Hg. The physician assessment of patient vision loss can be determined in many ways, for example with one or more of visual fields testing or the cup to disk ratio which is known measurement to assess the progression of glaucoma. The above treatment instructions may comprise menu selections of hand held physician device 330 that can be selected and forwarded to the hand held patient reader device 310.

The handheld communication device 330 may comprise a processor comprising a computer readable medium having instructions of a computer program embodied thereon so as to store and display patient data for diagnosis and treatment, for example data received from the server. The server located at the physician office may comprise a processor comprising a computer readable medium having instructions of a computer program embodied thereon so as to store patient data with the database. The remote server may comprise the server at the physician office.

The remote server 320 can be configured to communicate with processors of a community 350 of online users. The community 350 of online users may comprise a plurality of processors 352. The plurality of processors 352 may comprise, for example, a first user processor U1 of a first user, a second user processor U2 of a second user, a third user processor U3 of a third user and a fourth user processor U4 of a fourth user and an Nth user processor UN of an Nth user, for example a one millionth user. The online community 350 may comprise patients monitored with the implanted sensor device and friends, family members and care givers of the patients. The community of user may be connected with an online community social networking site comprising a vitualy community. For example the online community may comprise Facebook users.

The remote server 320 can be coupled to a community of remote online physicians 360 who can compare data and who can provide telemedecine to members of the online community 350. The community of remote online physicians can practice telemedecine with a patient, for example a patient of the community of users. The treating physician and physician device 330 may comprise a member of the community of remote online physicians 360. Each physician has access to a processor comprising a tangible medium having computer readable instructions stored thereon, for example a smartphone, a tablet computer, a notebook computer or a desk computer. For example a first processor TMD1 comprising a smart phone may be used by a first physician and a second processor TMD2 comprising a notebook computer may be used by a second physician.

The remote server 320 can control communication and access of the patient data, and may be configured to display information on the displays of the online community 350 and the processors of the community of remote online physicians. The remote server 320 can receive commands from the physician and transmit the treatment commands to the hand held external reader 310. For example, the physician can prescribe a target IOP for the patient based on the physician's evaluation of the patient, and the customized physician prescribed target IOP can be transmitted to the hand held external reader 310. The handheld external reader may comprise instructions of a computer program such that a message is transmitted to the treating physician, for example an email, when the patient IOP exceeds the customized prescribed target IOP. Alternatively or in combination, the remoter server may comprise instructions to transmit a message to the physician when the patient IOP exceeds the physician prescribed IOP for the patient.

Wireless Pressure Sensor

FIG. 3A shows components of an implantable MEMS pressure sensor for use with the implantable apparatus;

The pressure sensors may comprise many of types of known biocompatible pressure sensors sized for placement in the lens capsule.

The pressure transducer assembly may comprise a micro-electro-mechanical system (MEMS) and can be fabricated with known methods. The pressure sensor may be fabricated on a same substrate that can be coupled to the coil structure. The coil structure can be fabricated on a support separate and the pressure sensor, and the MEMS pressure sensor can be fabricated on a silicon substrate. The coil structure can be coupled to the pressure sensor in many ways. For example, the pressure sensor can be joined or attached to the coil structures with wires or with traces on a support such as a flex printed circuitry board. The pressure sensor may comprise a single chip sensor supported with a substrate, for example silicon or glass. The pressure sensor and substrate may be positioned on a flexible support as described above. A layer of silicon substrate can be oxidized to form a dielectric layer such as silicon dioxide (SiO₂, hereinafter “oxide”). A layer of conductive silicon semiconductor 370 can be deposited on the oxide 371 and shaped with lithography and etching so as to form a lower side of the capacitor. A layer of conductive metal such as gold can be deposited over the silicon and oxide so as to form a first trace 372 extending from the lower side 376 of the capacitor to a first end of the coil structure. A dielectric layer 375, for example SiO₂, can be deposited over the gold to separate the lower side of the capacitor from the upper side. A layer of conductive silicon semiconductor can be deposited on the dielectric layer opposite the lower side of the capacitor and shaped to form the upper side 374 of the capacitor. The upper side of the capacitor may comprise a sensing diaphragm that bends with pressure so as to decrease spacing of the first side of the capacitor from the second side such that the capacitance increases when pressure increases. A layer of conductor, for example gold, can be deposited on the second side of the capacitor comprising the pressure sensing diaphragm, and the conductor can be shaped to couple to form a second trace 373 to couple to the coil structure comprising the telemetric antenna.

Packaging

To protect the MEMS pressure sensor with wireless telemetry from corrosion, the implant device having the MEMS pressure sensor mounted thereon may be coated or encapsulated in a soft biocompatible polymer such as polydimethlysiloxane (PDMS). The MEMS pressure sensor may read pressure from all directions and may be covered with compliant enclosure filled with a conformable material such as liquid, viscous material, or gel (e.g., silicone, saline or other biocompatible material). This allows pressure to be uniformly exerted on the pressure sensor 130, such that pressure can be sensed from forces on a side opposite the pressure sensor. For example, the implant can be positioned such that the pressure sensor is located on a first side of the implant opposite a second side of the implant, and the device can measure pressure of the anterior chamber when the second side of the implant is positioned to contact tissue of the anterior chamber such as the cornea or iris and the first side of the implant is positioned away from the tissue in contact with the aqueous humor.

Antenna/Reader

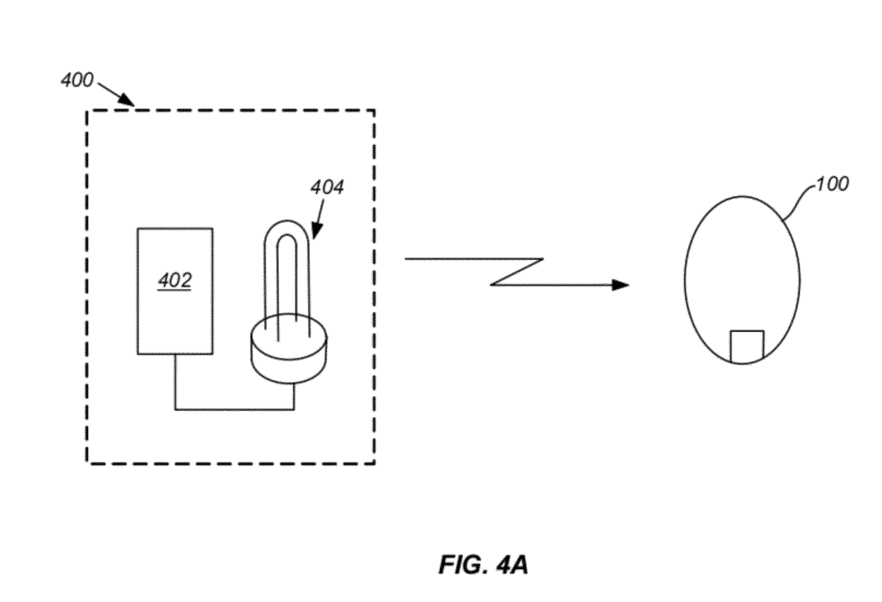

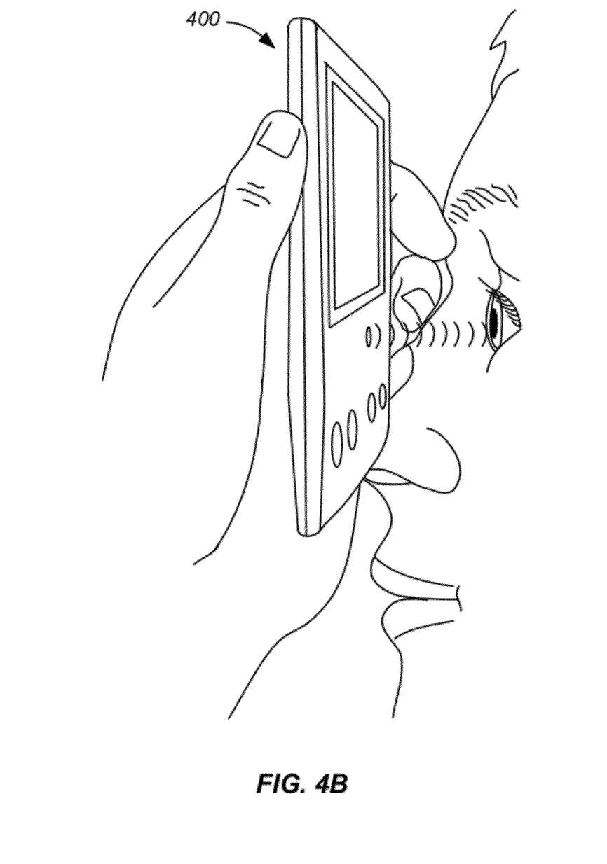

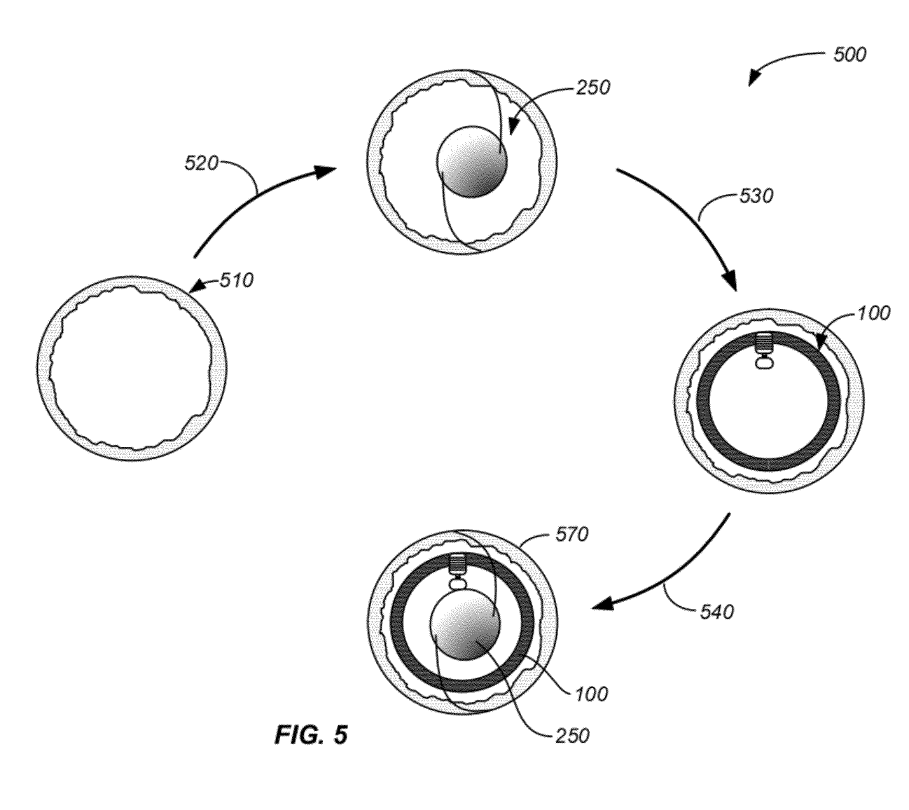

FIG. 4A shows components of an antenna/reader and processor 40 coupled to the antenna/reader to determine the IOP. The antenna/reader comprises circuitry 402 to emit a radio frequency signal with an antenna 404 so as interrogate the tank circuit of the implanted sensor device 100, such that the resonant frequency of the LC tank circuit can be determined. As the resonant frequency changes with pressure, the IOP measured with the sensor can be determined based on the resonant frequency. The reader can house the electronics and software, and may comprise a processor having a computer readable medium having instructions of a computer program embodied thereon so as to be used as a data collection, reporting and analysis platform, for example data mining to determine the presence of pressure spikes and trends. The processor can be programmed to measure the IOP and predetermined intervals or predetermined times, or both. The processor can be coupled to the Internet and the servers as described above.

FIG. 4B shows the hand held antenna/reader 400 with components similar to the antenna reader as in 4A. The hand held data reader may comprise the antenna, circuitry to determine the resonant frequency, and circuitry similar to a smart phone such as an iPhone™, such that the hand held reader can measure, store and transmit patient data.

FIG. 5 shows a method 500 of implanting a pressure sensor apparatus and an IOL during surgery such as cataract surgery. A step 510 prepares they eye for implantation of the IOL. For example the lens of the eye can be prepared in the same manner as for implantation of the IOL alone. An incision is made in the cornea of no more than about 3 mm, for example. The capsular bag can be cut and the cataract removed. Alternatively, with a phakic IOL, the capsular bag may not be cut such that the lens of the eye is preserved. A step 520 inserts the IOL with the IOL inserter as described above. A step 530 inserts the implantable sensor 100 as described above through the inserter as described above. The IOL placement can be in the anterior chamber, the posterior chamber, or in the capsule, and can occur either before or after the sensor placement in the anterior chamber, the posterior chamber, or the capsule. The order of implanting the IOL and the sensor can also be reversed so that the sensor is implanted first and the expandable ring can be in place to receive the IOL. A step 540 determines whether the IOL should be inserted before the sensor apparatus or after the sensor apparatus. For example, when the zonules of the eye are intact, the sensor apparatus can be implanted when the IOL has been placed such that the IOL provides a protective support for the sensor. When the zonules of the eye have been compromised, the sensor apparatus such as the C-shaped sensor device can be inserted before the IOL so as to expand and center the capsular bag with annular tensioning of the bag from the expanding c-ring structure of the sensor device. The sensor device 100 is shown placed with the IOL in the lens capsule.

It should be appreciated that the specific steps illustrated in FIG. 5 provide a particular method of treating a patient with an implantable pressure sensor apparatus implanted with an IOL during surgery such as cataract surgery, according to an embodiment of the present invention. Other sequences of steps may also be performed according to alternative embodiments. For example, alternative embodiments of the present invention may perform the steps outlined above in a different order. Moreover, the individual steps illustrated in FIG. 5 may include multiple sub-steps that may be performed in various sequences as appropriate to the individual step. Furthermore, additional steps may be added or removed depending on the particular applications. One of ordinary skill in the art would recognize many variations, modifications, and alternatives.

The processor system as described above can be configured to implement many of the steps of method 500. For example, the processor system may comprise a computer readable medium having instructions of a computer program embodied thereon to implement many of the steps of method 500.

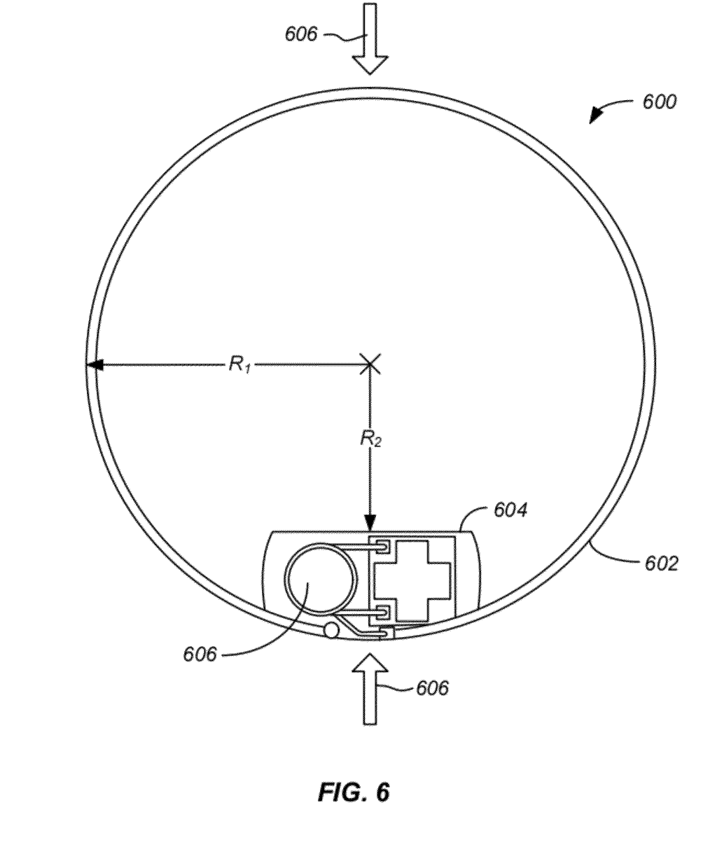

Referring to FIG. 6, an implantable sensor 600 in accordance with the present invention comprises a loop antenna (coil) 602 and a pressure sensing module 604. The loop antenna is formed from a nickel-titanium alloy and can be collapsed or “pinched” by applying pressure in the direction of arrows 606, eg using tweezers or another tool. By laterally collapsing the loop antenna 602, the width can be reduced to the width of the module (R₁−R₂), thus minimizing the profile which is inserted through the IOL or other tubular insertion tool. In the exemplary embodiment, the loop antenna comprises a paralyne-coated 76 μm nickel-titanium wire with 50 μm gold coating. The loop has a radius R, of 5 mm with a distance from center to the inner wall of the module 604 of about 3 mm (R₂), allowing the structure to be reduced to an approximately 2 mm profile for introduction. The limited 2 mm intrusion of the module 604 into the interior of the loop antenna is also advantageous as it facilitates placement of an IOL therein. A resonant coil 606 is provided to determine the intraocular pressure.

Experimental. A person of ordinary skill in the art can conduct experimental studies to determine empirically parameters of the implantable device, such that the device can be implanted for the extended time of at least one year, for example the diameter, the thickness, the curvature and flexibility of coil structure. Such studies can be conducted with an animal model, for example rabbits, and clinical studies with patients may also be conducted.

While the exemplary embodiments have been described in some detail, by way of example and for clarity of understanding, those of skill in the art will recognize that a variety of modifications, adaptations, and changes may be employed. It should be appreciated that the embodiments as described herein can be combined in many ways unless inconsistent with the teachings as described herein, and a person of ordinary skill in the art will recognize many combinations and adaptations based on the teachings as described herein. Further, combinations of embodiments may include more, or fewer elements, based on the teachings as described herein, and a person of ordinary skill in the art will recognize many adaptations and alternatives. Hence, the scope of the present invention shall be limited solely by the appended claims and the full scope of the equivalents thereof.

What is claimed is:

1. A method of treating an eye having a tissue structure, the method comprising:
inserting an implantable sensor device into the eye, the implantable sensor device having a pressure sensor and an electrically conductive coil structure coupled to the pressure sensor, wherein the electrically conductive coil structure engages a tissue structure of the eye to hold the implantable sensor device within the eye.

2. The method of claim 1, further comprising inserting a prosthetic lens in the eye wherein the sensor and the lens are inserted through the same incision.

3. A method of treating an eye having lens, the lens having a capsule, the method comprising:
inserting an intraocular lens (IOL) within the capsule, the IOL comprising a haptic to expand against the capsule; and
inserting an implantable sensor device within the capsule, the implantable sensor device having a pressure sensor and an electrically conductive coil structure coupled to the pressure sensor, wherein the electrically conductive coil structure engages an interior periphery of the capsule to hold the implantable sensor within the capsule.

4. The method of claim 3, wherein inserting the implantable sensor device comprises passing the device through a lumen of an IOL inserter to place the implantable sensor device within the capsule and wherein the implantable sensor device has an elongate narrow profile configuration when passed though the lumen and an expanded wide profile configuration when placed in the capsule.

5. The method of claim 3, wherein the electrically conductive coil structure comprises an elastic material so that it may be constrained while being introduced through the inserter and released to expand within the capsule.

6. The method of claim 5, wherein the pressure sensor comprises a variable capacitor having a capacitance responsive to pressure of the eye and wherein a resonant frequency of the variable capacitor corresponds to a pressure of the eye.

7. The method of claim 6, wherein the variable capacitor comprises a diaphragm variably spaced apart from a plate and wherein the expandable coil structure comprises a first end coupled to the diaphragm and a second end coupled to the plate.

8. The method of claim 6, wherein the expandable coil structure comprises an expandable curved loop structure and wherein the expandable curved loop structure is configured to expand radially outward toward the capsule.

9. The method of claim 8, wherein the expandable curved loop structure comprises a single loop having a first end coupled a first terminal of the pressure sensor and a second end coupled to a second terminal of the pressure sensor so as to define a closed area of the single loop extending between the first end and the second end.

10. The method of claim 8, wherein the expandable loop structure has an elongate conductor and wherein the elongate conductor and the sensor encloses a substantially constant area so as to maintain a resonant frequency of the expandable loop structure and the sensor when the loop structure expands radially outward toward the capsule.

11. The method of claim 10, the curved loop structure comprises a first curved portion and a second curved portion and a support extending between the first curved portion and the second curved portion so as to maintain the substantially area of the loop structure when the loop structure expands from a first radial size to a second radial size.

12. The method of claim 11, wherein the expandable loop structure comprises a C-shaped configuration.

13. The method of claim 1, wherein the expandable loop structure applies a radially outward tension to the capsule when placed inside the capsule.

14. The method of claim 8, wherein the expandable electrically conductive loop structure comprises undulations to increase a dimension across the electrically conductive loop structure.

15. The method of claim 3, wherein the coil structure comprises a toroidal coil and a loop structure coupled to the pressure sensor to determine a pressure of the eye based on a resonant frequency of the loop structure and the toroidal coil coupled to the pressure sensor.

16. The method of claim 3, wherein the implantable sensor device is positioned within the lens capsule prior to placement of the IOL and wherein the implantable sensor device tensions the capsular bag and aligns the IOL with a visual axis of the eye when the IOL is placed in the capsular bag.

17. The method of claim 3, wherein the implantable sensor device is positioned within the lens capsule after placement of the IOL.

18. A device for measuring an intraocular pressure of an eye having a lens, the lens having a tissue structure, the device comprising:
a pressure sensor; and
an expandable electrically conductive coil structure coupled to the pressure sensor, wherein the expandable coil structure comprises a resilient material to urge the coil structure against the tissue structure to hold the coil structure and the pressure sensor within the eye.

19. The device of claim 18, wherein the expandable coil structure is adapted to urge against a capsule of the lens when placed in the capsule so as to center the coil structure about an optical axis of the eye.

20. The device of claim 18, wherein the coil structure is capable of expanding to a maximum dimension across of at least about 13 mm.

21. The device of claim 18, wherein the expandable electrically conductive coil structure and the pressure sensor are capable of passing through a lumen of an IOL inserter having a diameter of no more than about 3 mm.

22. The device of claim 18, wherein the expandable electrically conductive coil structure comprises an expandable curved loop structure.

23. The device of claim 22, wherein the expandable curved loop structure comprises a single loop having a first end coupled a first terminal of the pressure sensor and a second end coupled to a second terminal of the pressure sensor so as to define a closed area of the single loop extending between the first end and the second end.

24. The device of claim 22, wherein the expandable loop structure comprises one or more of an expandable support or an expandable wire structure having a first elongate narrow profile configuration to pass through an incision in a cornea of the eye of no more than about 3 mm across and wherein the expandable coil structure comprises an expanded wide profile configuration to fit within the capsule.

25. The device of claim 24, wherein the expandable loop structure comprises the expandable wire structure, the expandable wire structure having a wire and a soft covering material disposed over the wire to allow the wire to expand and urge the soft covering material against the capsule.

26. The device of claim 25, wherein the resilient metallic conductor comprises a shape memory alloy composed of one or more of copper-zinc-aluminum-nickel, copper-aluminum-nickel, nickel-titanium, zinc, copper, aluminum, nickel, titanium, gold or, iron.

27. The device of claim 24, wherein the expandable loop structure comprises the expandable support, the expandable support comprising a resilient material to urge outward against the capsule when placed within the capsule.

28. The device of claim 27, wherein the resilient material comprises one or more of a polyimide, an acrylate, a soft acrylate, a polymethylmethacrylate (PMMA), a silicone, or a shape memory polymer.

29. The device of claim 22, wherein the expandable loop structure comprises a first end separated from a second end and wherein a distance between the first end and the second end corresponds to a radial size of the loop structure.

30. The device of claim 29, wherein the pressure sensor is located on the first end of the expandable loop structure and wherein an inductance of the loop structure remains substantially constant when the first end moves away from the second end to tension the capsular bag with the annular loop structure.

31. The device of claim 29, wherein the expandable loop structure comprises an elongate support having a trace of electrical conductor deposited thereon and coupled to the pressure sensor so as to define an inductive area with the trace of electrical conductor coupled to the pressure sensor.

32. The device of claim 31, wherein the area comprises a substantially constant area when the radial size of the loop structure expands from a first size to a second size.

33. The device of claim 32, wherein the area extends circumferentially away from the optical axis of the eye and around an optical axis of the eye and, and wherein the first size corresponds to a first dimension transverse to the optical axis of the eye and the second size corresponds to a second dimension transverse to the optical axis of the eye when the loop structure is positioned in the capsule.

34. The device of claim 29, wherein the expandable loop structure comprises a C-shaped configuration.

35. The device of claim 22, wherein the expandable loop structure comprises a substantially annular loop structure having at least an expandable portion sized and shaped to expand from a first annular size to a second annular size to fit the capsule when placed within the capsule.

36. The device of claim 35, wherein the substantially annular portion comprises an annular ring.

37. The device of claim 35, wherein the substantially annular portion comprises an inner radius and an outer radius.

38. The device of claim 37, wherein the inner radius and the outer radius increase together to maintain the substantially constant area.

39. The device of claim 37, wherein the inner radius comprises at least about 3 mm and the outer radius comprises at least about 1 mm more than the inner radius such that an area defined with the inner radius and the outer radius comprises at least about 5 square mm.

40. The device of claim 39, wherein the inner radius comprises at least about 4 mm and the outer radius comprises at least about 1.0 mm more than the inner radius such that an area defined with the inner radius and the outer radius comprises at least about 10 square mm.

41. The device of claim 35, wherein the expandable portion comprises one or more of a coil, a serpentine pattern, undulations or slack in an elongate electrical conductor to increase a circumference of the substantially annular loop structure when the loop structure expands from the first annular size to the second annular size.

42. The device of claim 35, wherein the substantially annular portion comprises one or more of an oval annular portion, an elliptical annular portion, or a circular annular portion.

43. The device of claim 18, wherein the resilient material comprises one or more of a metal, a shape memory material, a shape memory metal, nitinol, a plastic, a soft plastic, a soft acrylate, a PMMA or a silicone.

44. The device of claim 18, wherein the expandable coil structure has a single loop of a first electrical conductor coupled to a toroidal coil having a plurality of turns and wherein an intraocular pressure of the eye is determined based on a resonant frequency of the pressure sensor coupled to the single loop and the toroidal coil.
